(19) United States
(12) Patent Application Publication
(10) Pub. No.: US 2014/0377793 A1
(43) Pub. Date: Dec. 25, 2014

(54) Device And Method Of Sampling And Analysing Biological Or Biochemical Species

(71) Applicant: Commissariat A L'Energie Atomique et aux Energies Alternatives, Paris (FR)

(72) Inventors: Ali Bouamrani, Grenoble (FR); François Berger, Meylan (FR); Marie-Line Cosnier, Grenoble (FR)

(21) Appl. No.: 14/369,234

(22) PCT Filed: Dec. 17, 2012
(86) PCT No.: PCT/IB2012/057387

§ 371 Date: Jun. 27, 2014

(30) Foreign Application Priority Data
Dec. 29, 2011 (FR) 1162530

(51) Int. Cl. G01N 27/62; A61B 10/02; A61B 10/00

(52) U.S. Cl. CPC G01N 27/62; A61B 10/007; A61B 10/0064; A61B 10/02
USPC 435/29; 600/562; 435/287.1

(57) ABSTRACT

A method of sampling biological or biochemical species, comprising the following steps: a) arranging a capture surface (SC) for said biological or biochemical species in contact with a biological tissue or fluid (TB); and b) rinsing said surface to remove biological or biochemical species that have not been adsorbed; the method being characterised in that said capture surface is the surface of a nanoporous material (MC). A method of analysing said biological or biochemical species, characterised by the use of said surface as an analysis support. The analysis may in particular be performed using a method selected from mass spectroscopy with laser desorption and fluorescence imaging. A device for sampling biological or biochemical species comprising a rod (TM) to which is attached a material (MC) having a capture surface (SC) for said biological or biochemical species, arranged so as to be able to be brought into contact with a biological tissue or fluid (TB), characterised in that said material is a nanoporous material. The rod can be slid into a guide tube to facilitate the insertion thereof into a human or animal body.

The invention relates to a method of sampling biological or biochemical species, to a method of analyzing biological or biochemical species and to a device for carrying out these methods.

One of the priorities in biomedical research is to improve anatomicopathologic, histological and molecular analyses. The conventional histological approach is based on biopsy techniques for collecting samples of biological tissues. These techniques are still relatively intrusive, despite the progress made possible by miniaturization of the devices employed. Moreover, the procedures for preparing the tissues (fixation in paraffin, freezing, etc.) are not very compatible with the new approaches in molecular investigation. The fragility of biochemical species such as RNA and proteins is one of the pre-analytical factors explaining the relative failure of clinical transfer of the innovative “poly-omic” approaches.

Moreover, owing to their cost and the time required for their execution, the current procedures are poorly compatible with the need for extemporaneous analyses, i.e. carried out in the operating theater during surgery, in order to aid the surgeon's decisions. Moreover, histological analysis using rapid staining does not always allow sufficiently relevant information to be obtained.

Document WO 2006/082344 describes a device for molecular sampling by contact comprising a support having a face that is structured at the micrometric scale, for example in the form of a network of micro-studs, or else of micro-cuvettes that may be filled with micro-beads. This face, which is preferably functionalized, has a relatively large developed surface, able to capture by simple contact and fix molecules of interest contained in a biological tissue. The use of functionalization of the surface introduces constraints in terms of preservation of the device during storage and in terms of sterilization (as the conventional methods of sterilization are likely to affect the functionalization). In the absence of functionalization, the efficacy and selectivity of capture are inadequate.

The invention aims to overcome these drawbacks of the prior art. According to the invention, this result is achieved through the use of a nanoporous contact surface, in particular made of nanoporous silicon. The nanopores allow efficient and relatively selective adsorption of chemical and biological species, even in the absence of surface functionalization (although functionalization may also be envisaged in certain embodiments, to further improve the selectivity and/or efficacy of capture); this is attributed to a suction effect by the pores. Document WO 2011/025602 discloses the use of nanoporous materials—and notably nanoporous silica—for the fractionation, stabilization and storage of biomolecules.

One object of the invention is therefore a method of sampling biological or biochemical species comprising the following steps:

a) arranging a surface for capturing said biological or biochemical species in contact with a biological tissue or fluid, in such a way that at least one biological or biochemical species is adsorbed by said surface; and

b) rinsing said surface to remove the biological or biochemical species that have not been adsorbed;

in which said step a) is not of the nature of surgery;

the method being characterized in that said capture surface is the surface of a nanoporous material.

The biological tissue may notably be a tissue other than a liquid tissue such as blood. It may for example be an epithelium, and notably an endothelium, or a connective tissue. The biological fluid (generally a liquid) may notably be whole blood, blood plasma, cerebrospinal fluid, saliva, etc. It may be human, animal (nonhuman) and/or vegetable tissues or fluids.

“Nanoporous material” means a crystalline or amorphous material, all in one piece and preferably of homogeneous composition, having pores whose average diameter is less than a micrometer and in particular less than or equal to 100 nm. Among the nanoporous materials, a distinction is notably made between materials that are microporous (pores with an average diameter between 0.2 and 2 nm), mesoporous (pores with an average diameter between 2 and 50 nm) and macroporous (pores with an average diameter between 50 and 1000 nm). The porosity (ratio of pore volume to total volume) will preferably be greater than or equal to 10%.

The biological species adsorbed may be cells (diameter between about 1 μm and 50 μm), bacteria, viruses, and circulating vesicles such as exosomes (diameter between about 20 and 200 nm). The biochemical species adsorbed may be molecules or macromolecules, such as proteins (diameter from some nanometers to some tens of nanometers), peptides (size of the order of a nanometer), and metabolites. The “size” of these molecules is to be understood as their largest dimension.

It is considered that an operation is not of the nature of surgery when it is not intrusive, or whenever it does not represent a substantial physical intervention on the body, does not require professional medical expertise and does not pose a substantial health risk. In general, any operation consisting of putting the capture surface in contact with the epidermis, a tissue accessible via natural passages or openings (rectum, oral cavity, urethra, bladder, etc.), or else by simple penetration of the epidermis, even by an intravenous catheter, is not of the nature of surgery. Bringing the capture surface into contact with a tissue made accessible by a previous surgical operation (for example, introduction of a catheter, when this operation is of a surgical nature) or one that is concomitant, but carried out independently, also is not of the nature of surgery.

The method may also comprise a step c) consisting of analyzing the biological or biochemical species adsorbed by said capture surface, the latter being used as an analysis support. In particular, said step c) may be carried out by a method selected from mass spectroscopy with laser desorption and an imaging technique such as fluorescence imaging. Notably, said step c) may be carried out by a method of mass spectroscopy with laser desorption of the MALDI (matrix-assisted laser desorption/ionization) or SELDI (surface enhanced laser desorption/ionization) type, an organic matrix being deposited directly on the capture surface after said rinsing step b). As a variant, step c) may also be carried out by a method of Raman scattering spectrometry.

The fact that the capture material may serve as the analysis support constitutes a particularly advantageous feature of the invention. In fact, transfer from the capture surface to a separate analysis support could damage the biochemical or biological species captured, which are often very fragile. Moreover, this transfer would be a source of complexity, of costs and of risks of contamination or error. By comparison, in the case of the aforementioned document WO 2011/025602, an elution step is necessary for being able to analyze the captured and stabilized molecules.

According to a preferred embodiment of the method, said nanoporous material may be nanoporous silicon. Advantageously, said nanoporous silicon may have at least one of the following properties (and preferably all of these properties):
pores of dendritic structure;
pores with an average diameter between 1 and 100 nm; and
a porosity between 40% and 65% to a depth between 10 nm and 100 μm.

Preferably, said capture surface is not functionalized.

Said step a) may be carried out ex vivo, on a sample of biological tissue or fluid previously taken from a human, animal or vegetable organism; preferably, it could be a “fresh” tissue, i.e. that has not undergone a treatment of freezing or fixation. As a variant, said step a) may be carried out in vivo, provided that this does not involve a substantial physical intervention on the body requiring medical expertise and posing a substantial health risk for a patient—i.e. provided that this does not involve an intervention of a surgical nature.

Another object of the invention is a method of analyzing biological or biochemical species previously adsorbed on the surface of a nanoporous material, characterized by the use of said surface as the analysis support. In particular, said analysis may be carried out by a method selected from mass spectroscopy with laser desorption and an imaging technique such as fluorescence imaging. Notably, said analysis may be carried out by a method of mass spectroscopy with laser desorption of the MALDI (matrix-assisted laser desorption/ionization) or SELDI (surface enhanced laser desorption/ionization) type, using an organic matrix deposited directly on the surface of the nanoporous material. It may also be imaging techniques, such as fluorescence imaging or colorimetry, and these analyses may be preceded by addition of a suitable marker to the medium. It may also be an analysis by Raman scattering spectrometry.

According to a preferred embodiment of the method, said nanoporous material may be nanoporous silicon. Advantageously, said nanoporous silicon may have at least one of the following properties (and preferably all of these properties):
pores of dendritic structure;
pores with an average diameter between 1 and 100 nm; and
a porosity between 40% and 65% to a depth between 10 nm and 100 μm.

Preferably, said capture surface is not functionalized.

Yet another object of the invention is a device for sampling biological or biochemical species comprising a rod, to which a material is fixed that has a capture surface for said biological or biochemical species, arranged so that it can be brought into contact with a biological tissue or fluid, characterized in that said material is a nanoporous material.

According to a preferred embodiment of the device, said nanoporous material may be nanoporous silicon.

Advantageously, said nanoporous silicon may have at least one of the following properties (and preferably all of these properties):
pores of dendritic structure;
pores with an average diameter between 1 and 100 nm; and
a porosity between 40% and 65% to a depth between 10 nm and 100 μm.

Preferably, said capture surface is not functionalized.

Said rod may be placed inside a guide tube having a lateral or axial opening, and said rod can be moved within said guide tube so as to bring said capture surface into correspondence with said opening. In particular, said rod may have a flat surface to which said material is fixed and said guide tube may have a lateral opening, and the capture surface can be brought opposite said opening by rotation of the rod within the guide tube.

Other features, details and advantages of the invention will become clear on reading the description, referring to the appended drawings, given as an example, in which:

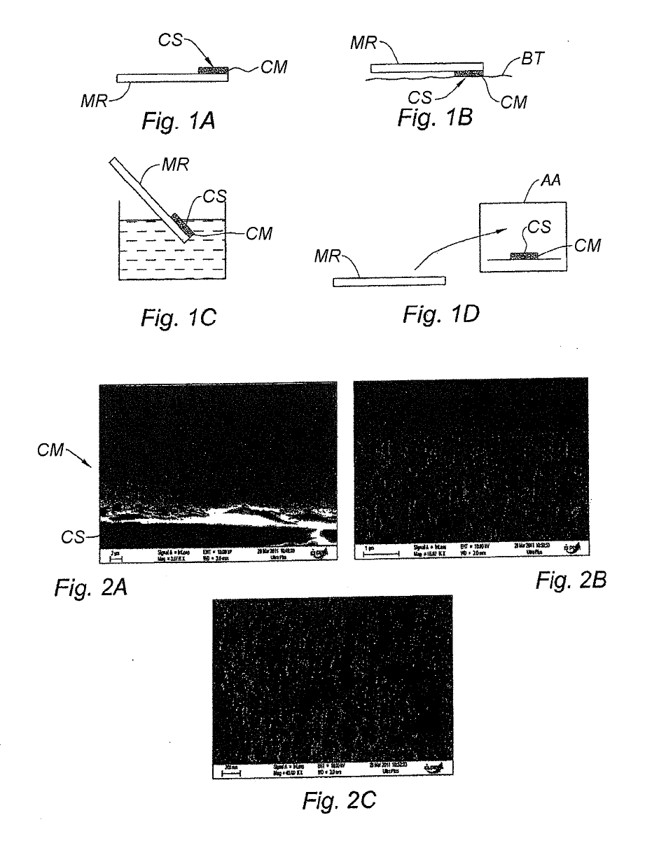

FIGS. 1A-1D illustrate schematically a device and a method according to the invention;

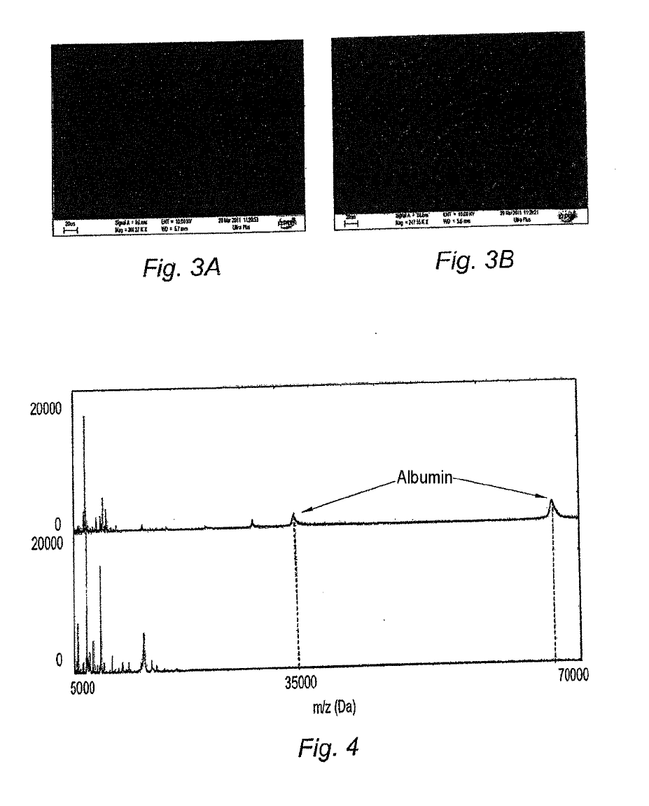

FIGS. 2A-2C and 3A, 3B show views with the electron microscope, respectively of the slice and of the surface, of various samples of nanoporous silicon that may be suitable for carrying out the invention;

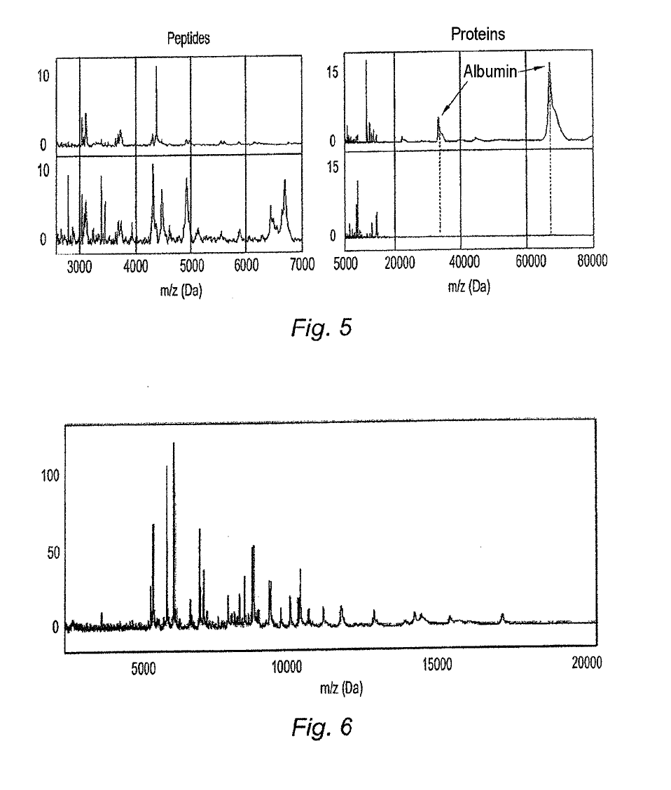

FIGS. 4, 5 and 6 show mass spectra of proteins obtained on applying a method according to one embodiment of the invention for investigating: human blood plasma, human cerebrospinal fluid and mouse brain tissue, respectively;

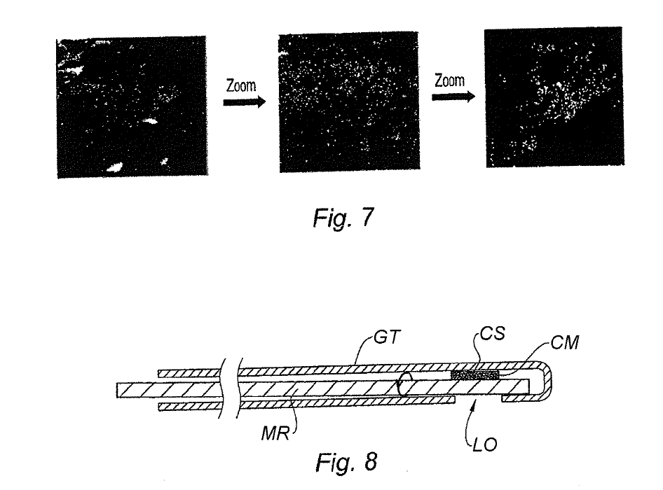

FIG. 7 shows fluorescence images demonstrating the capture of cells from mouse brain tissue using a device according to the invention; and

FIG. 8 illustrates a device according to one embodiment of the invention.

FIG. 1A illustrates schematically a device according to one embodiment of the invention, consisting of a plate CM of a capture material, of the nanoporous type, fixed—preferably detachably—on a manipulating rod MR. The plate of capture material has, opposite its surface for fixing to the rod MR, a nanoporous capture surface CS, typically having a size between 0.5 and 5 cm². The capture material may be nanoporous silicon, and more particularly mesoporous.

As illustrated in FIG. 1B, the rod MR is used for bringing the capture surface CS into contact with a biological tissue BT. It may be a sample of tissue previously taken from a human or animal body, or even from a vegetable organism (ex vivo), or else from a tissue in vivo. Contacting may be effected by simple bringing together, without it being necessary to rub the tissue or apply increased pressure. This is important, especially for applications in vivo.

As a variant, the capture material could be immersed in a biological fluid, or a droplet of said fluid could be deposited on the capture surface.

The capture surface of nanoporous silicon is smooth to the touch and does not have significant asperities. Thus, the risk of lesion of the tissue is minimized, which is very advantageous for applications in vivo. Therefore the sampling of the biological or biochemical species is not carried out by micro-abrasion, nor by chemical functionalization, but by a suction effect due to the nanopores. This effect leads to preferential fixation of the peptides and of the “small” proteins, having a size of the order of about 1 to 5 nm, and/or a mass between about 5000 and 20 000 Da. This is advantageous, as these “small” proteins are generally more useful as markers than larger molecules. The suction effect also explains the adhesion of the cells, which are too large to be able to penetrate into the pores.

Next (FIG. 1C), the device is removed from the tissue, and the capture surface is rinsed, for example by immersion in water, or using a buffer solution, which makes it possible to remove the biological or biochemical species that have not been adsorbed by the surface, and therefore are not necessarily adhering to it. This rinsing makes it possible to reveal the selective character of adsorption by the nanoporous surface.

Finally, the plate CM of capture material is separated from the rod (although this is not always necessary) and introduced into an analyzer AA, for analyzing the biological or biochemical species adsorbed by the capture surface. Typically, the analyses can be carried out by mass spectroscopy with laser desorption, of the MALDI or SELDI type. In this case, it will be necessary to deposit a suitable organic matrix on the capture surface. As a variant or in addition, it will be possible to perform analyses by fluorescence imaging. In any case, the capture material CM serves directly as the analysis support. For the analyses with laser desorption and ionization, it is necessary for the capture material to be conductive—which is the case with doped nanoporous silicon.

As mentioned above, the capture material used is preferably nanoporous silicon, although it is perfectly possible to use other nanoporous materials.

Nanoporous (and more precisely mesoporous) silicon may be obtained by electrochemical anodization of p+ doped silicon with conductivity from 10 to 20 mΩ·cm in a solution of hydrofluoric acid at about 15%. For this purpose, the material is immersed in a bath of HF and is submitted to electrolysis, which is a known process. In this way, a material is obtained having a porosity with a dendritic structure; this signifies that the pores do not have a rectilinear axis; they extend in the depth of the material in a discontinuous direction, and may cross. This dendritic structure promotes suction. The porosity (ratio of pore volume to total volume) is between 40 and 65%. The porosity extends to a depth of about 6 μm. Beyond that, there is massive silicon. Generally, in this invention, the porosity of the material may extend to a depth that may be between 10 nm and 100 μm.

These characteristics may easily be varied by varying the manufacturing parameters (concentration of HF, anodization time, current density, type of silicon).

As a variant, it is possible to produce nanoporous silicon having an ordered structure by a process of electronic lithography.

FIGS. 2A, 2B and 2C show electron microscopy images of the slice of a sample of nanoporous silicon obtained by the electrochemical anodization process described above. The figures have different magnifications; in particular, FIG. 2B demonstrates the clear transition between massive silicon and nanoporous silicon, whereas FIG. 2C shows the dendritic structure of the pores.

FIGS. 3A and 3B show electron microscopy images of the surface of two samples of nanoporous silicon obtained by electrochemical anodization in different conditions.

The device and the method of the invention were tested by means of three tests ex vivo.

In a first test, 5 μl of human blood plasma were deposited directly on the capture surface made of nanoporous silicon. Then the surface was rinsed twice using an acidic buffer (sodium acetate 100 mM, pH 4.0) for 1 min. As silicon has a slightly negatively charged surface charge, a buffer solution is used with its pH adjusted to promote a positive charge of the proteins of the sample that we wish to collect. In other words, the pH is adjusted in relation to the isoelectric point of the proteins of interest. Note that the isoelectric point of a protein corresponds to the pH for which the electric charge of said protein is zero. This rinsing makes it possible to remove the species that have not adhered to the capture surface, or impurities (residues of tissue, blood, etc.). Then the surface was rinsed once again in water, then it was dried in the air. An organic matrix (sinapinic acid) was deposited on the capture surface, which was then submitted to a MALDI analysis using a commercial MALDI mass spectrometer (Bruker Autoflex). Automatic acquisition of the spectra was carried out on 5400 laser impacts distributed regularly for each sample. The analysis was performed in linear mode (which means that the proteins describe a linear trajectory in the mass spectrometer) with an intensity of 55 (setting of the apparatus), with attenuation of the matrix signal at 1000 Da.

The results are shown in FIG. 4, where m/z is the mass/charge ratio (in dalton/elementary units of charge) and the abscissa shows the intensity of the mass spectroscopy signal (arbitrary units). The bottom graph corresponds to the use of nanoporous material, according to the invention; the top graph serves as reference and was obtained using a substrate holder of the type normally used in mass spectrometry (smooth metal strip on which a polymer is deposited), the reference of which is Biorad CM10. The measurements on the reference support were carried out according to the same protocol as those on the surface of nanoporous silicon.

It is observed that the species characterized by a ratio m/z>10000—and notably albumin, an abundant constituent of little interest for establishing a diagnosis—are not detected when a capture surface of nanoporous silicon is used, which demonstrates the selectivity of capture. Conversely, an enrichment of the peaks corresponding to the proteins of low mass is observed; in the case of the smooth support, in contrast, these proteins are largely removed by the rinsing.

It is also observed that the spectra are richer on the peaks corresponding to proteins of low mass, which confirms good adhesion of the small proteins on the nanoporous surface, despite the rinsing operations. On the smooth control support, the small proteins are largely removed by the rinsing.

In a second test, 10 μl of human cerebrospinal fluid were deposited directly on the capture surface of nanoporous silicon, of the same type as used for the first test. Then the surface was rinsed twice using an acidic buffer (sodium acetate 100 mM, pH 4.0) for 1 min. This rinsing makes it possible to remove the species that have not adhered to the capture surface, or impurities (residues of tissue, blood, etc.). Then the surface was rinsed once again in water, then it was dried in the air. An organic matrix (sinapinic acid for analysis of the proteins, CHCA, i.e. α-cyano-4-hydroxycinnamic acid, for analysis of the peptides) was deposited on the capture surface, which was then submitted to an analysis by means of a commercial SELDI mass spectrometer (Biorad PCS 4000).

The reading parameters were adjusted as a function of the scale of mass of the species to be detected. The optimum conditions were determined manually on some spots before starting automatic acquisition on 583 laser impacts distributed regularly for each sample:

An intensity of 1000 nJ and an attenuation of the matrix signal at 500 Da were used for the peptides (low molecular weights).

An intensity of 2200 nJ and an attenuation of the matrix signal at 1000 Da were used for the proteins (high molecular weights).

As in the first test, the same analysis was also carried out using a Biorad CM10 smooth substrate holder.

The results are shown in FIG. 5, where the top graphs were obtained with the smooth reference substrate and the top graphs were obtained with a nanoporous capture surface, according to the invention. Regarding the proteins, the results of the first test are confirmed: the “small” proteins are fixed effectively whereas substantial removal of the “large” proteins, and notably of albumin, is observed. The difference between the two supports is even greater in the case of the peptides: these are largely removed by rinsing in the case of the smooth support, whereas a very rich spectrum is observed in the case of the nanoporous support. The measurements on the reference support were carried out according to the same protocol as those on the surface of nanoporous silicon.

In a third test, a sample of fresh mouse brain tissue was put on the capture surface of nanoporous silicon, of the same type as that used for the first and the second test. Then the surface was rinsed twice using an acidic buffer (sodium acetate 100 mM, pH 4.0) for 1 min. This rinsing makes it possible to remove the species that have not adhered to the capture surface, or impurities (residues of tissue, blood, etc.). Then the surface was rinsed once again in water, then it was dried in the air.

The cells captured were detected by fluorescence imaging, owing to addition of a DNA intercalating agent (Hoechst buffer)—see FIG. 7. This figure reveals the presence of cells on the capture surface after rinsing.

An organic matrix (sinapinic acid) was deposited on the capture surface, which was then submitted to an analysis using a commercial SELDI mass spectrometer (Biorad PCS 4000). The results are shown in FIG. 4, where m/z is the mass/charge ratio (in dalton/elementary units of charge) and the abscissa shows the intensity of the mass spectroscopy signal (arbitrary units). Once again, a very rich mass spectrum is observed in the region 5000-10000 Da.

FIG. 8 (which is not to scale) illustrates an embodiment of a device of the invention that is particularly suitable for applications in vivo.

In this device, the rod MR is flexible and has a diameter of 500 μm and a length of 20 cm. About 1 cm from its end there is a flat surface with length of 2 cm, where the capture material CM is fixed, detachably. The rod slides in a guide tube or catheter GT made of a biocompatible material, having an outside diameter of 1 mm and a wall with a thickness of 50 μm. The distal end of the tube is closed; at a distance of about 1 cm from the latter, a lateral opening LO is made on a length of 2 cm. Initially, as illustrated in the figure, the rod is arranged in such a way that the capture surface CS is away from the opening. The guide tube with the rod is introduced into a patient's body, until the opening LO is opposite the tissue to be analyzed. A 180° rotation of the rod about its axis within the tube makes it possible to bring the capture surface CS into correspondence with said opening, and therefore in contact with the tissue. Another 180° rotation brings the capture surface to its initial position. Then the guide tube assembly is withdrawn from the patient's body, the capture material is separated from the rod and is submitted to the steps of rinsing and analysis as described above. As a variant, the guide tube may have been introduced into the patient's body beforehand; in this case, it is only necessary to introduce the rod into the tube, effect the double rotation and withdraw it.

As a variant, the tube may have an axial opening, at its distal end. In this case, the capture surface is brought into contact with the tissue by a forward movement of the rod.

It is to be understood that other devices may be used for carrying out an analysis in vivo according to the invention.

1. A method of sampling and analyzing biological or biochemical species comprising the following steps:
a) arranging a capture surface of said biological or biochemical species in contact with a biological tissue or fluid, in such a way that at least one biological or biochemical species is adsorbed by said surface, said capture surface being the surface of a nanoporous material; and
b) rinsing said surface to remove the biological or biochemical species that have not been adsorbed;
in which said step a) is not of the nature of surgery;
the method being characterized in that it also comprises a step:
c) consisting of analyzing the biological or biochemical species adsorbed by said capture surface, the latter being used as analysis support.

2. The method as claimed in claim 1, wherein said step c) is carried out by a method selected from:
mass spectroscopy with laser desorption; and
an imaging technique, such as fluorescence imaging.

3. The method as claimed in claim 2, wherein said step c) is carried out by a method of mass spectroscopy with laser desorption of the MALDI or SELDI type, an organic matrix being deposited directly on the capture surface after said rinsing step b).

4. The method as claimed in claim 1, wherein said nanoporous material is nanoporous silicon.

5. The method as claimed in claim 4, wherein said nanoporous material is nanoporous silicon having at least one of the following properties:
pores of dendritic structure;
pores with an average diameter between 1 and 100 nm; and
a porosity between 40% and 65% to a depth between 10 nm and 100 μm.

6. The method as claimed in claim 1, wherein said capture surface is not functionalized.

7. The method as claimed in claim 1, wherein said step a) is carried out ex vivo, on a sample of biological tissue or fluid previously taken from a human, animal or vegetable organism.

8. The method as claimed in claim 1, wherein said step a) is carried out in vivo, without involving a substantial physical intervention on the body that requires medical expertise and poses a substantial health risk for a patient.

9. A method of analyzing biological or biochemical species previously adsorbed on the surface of a nanoporous material, comprising a step of employing said surface as analysis support.

10. The method as claimed in claim 9, wherein said analysis is carried out by a method selected from:
mass spectroscopy with laser desorption; and
an imaging technique, such as fluorescence imaging.

11. The method as claimed in claim 10, wherein said analysis is carried out by a method of mass spectroscopy with laser desorption of the MALDI or SELDI type using an organic matrix deposited directly on the surface of the nanoporous material.

12. The method as claimed in claim 9, wherein said nanoporous material is nanoporous silicon.

13. The method as claimed in claim 12, wherein said nanoporous material is nanoporous silicon having at least one of the following properties:
pores of dendritic structure;
pores with an average diameter between 1 and 100 nm; and
a porosity between 40% and 65% to a depth between 10 nm and 100 μm.

14. The method as claimed in claim 9, wherein said surface is not functionalized.

15. A device for sampling biological or biochemical species comprising a rod to which a material is fixed having a capture surface of said biological or biochemical species, arranged so that it can be brought into contact with a biological tissue or fluid, characterized in that said material is a nanoporous material.

16. The device as claimed in claim 15, wherein said nanoporous material is nanoporous silicon.

17. The device as claimed in claim 16, wherein said nanoporous material is nanoporous silicon having at least one of the following properties:
pores of dendritic structure;
pores with an average diameter between 1 and 100 nm; and
a porosity between 40% and 65% to a depth between 10 nm and 100 μm.

18. The device as claimed in claim 15, wherein said capture surface is not functionalized.

19. The device as claimed in claim 15, wherein said rod is placed inside a guide tube having a lateral opening or axial opening, said rod being movable within said guide tube so as to bring said capture surface into correspondence with said opening.

20. The device as claimed in claim 19, wherein said rod has a flat surface to which said material is fixed and said guide tube has a lateral opening, and the capture surface can be brought opposite said opening by rotation of the rod within the guide tube.
